(19) United States
(12) Patent Application Publication
(10) Pub. No.: US 2022/0296648 A1
(43) Pub. Date: Sep. 22, 2022

(54) COMPOSITION FOR REGENERATING NUCLEUS PULPOSUS

(71) Applicant: AJOU UNIVERSITY INDUSTRY-ACADEMIC COOPERATION FOUNDATION, Suwon-si (KR)

(72) Inventor: Byoung-Hyun MIN, Anyang-si (KR)

(73) Assignee: AJOU UNIVERSITY INDUSTRY-ACADEMIC COOPERATION FOUNDATION, Suwon-si (KR)

(21) Appl. No.: 17/627,905

(22) PCT Filed: Jul. 13, 2020
(86) PCT No.: PCT/KR2020/009182

§ 371 Date: Jun. 2, 2022

(30) Foreign Application Priority Data
Jul. 17, 2019 (KR) 10-2019-0086411

(51) Int. Cl. A61K 35/32; A61L 27/36; A61P 19/00

(52) U.S. Cl. CPC A61K 35/32; A61L 27/3612; A61L 27/3658; A61P 19/00

(57) ABSTRACT

The present invention provides a composition for regenerating nucleus pulposus and a composition for regenerating an intervertebral disc tissue, each comprising fetal cartilage tissue-derived cells and a fetal cartilage tissue-derived extracellular matrix as active ingredients, and a pharmaceutical composition comprising the composition as an active ingredient for preventing or treating a vertebral disease. Simulating features of the nucleus pulposus, such as compression and tensile strength, the composition for regenerating nucleus pulposus can be regenerated into a tissue similar thereto. The composition for regenerating nucleus pulposus can be implanted into an intervertebral disc to restore an injured intervertebral disc tissue. Hence, the pharmaceutical composition comprising same for preventing or treating a vertebral disease can be used as a basic therapeutic agent for a vertebral disease such as a herniated intervertebral disc.

### TECHNICAL FIELD

The present disclosure relates to a composition for regenerating nucleus pulposus.

### BACKGROUND ART

Low back pain is so common that 84% of the population will experience it at least once in their lifetime. Low back pain caused by degenerative changes in an intervertebral disc is common in more than 80% of the population, and the degenerative changes are due to a decrease in a partial pressure of moisture in nucleus pulposus by a decrease of nucleus pulposus cells, and reduced production of extracellular matrix (ECM) such as a glycoprotein and a type II collagen. As a result, general degenerative changes occur in a facet joint and a vertebral body, which results in severe pain and limited activity in the patient. The degenerative changes of the intervertebral disc are caused by various factors such as genetic factors, environmental factors, and aging, and a chronic weight load, an intense physical activity, and obesity contribute to accelerating the degenerative changes of the intervertebral disc.

According to the National Health Insurance Service, as a result of analyzing health insurance medical expenses payment data from 2006 to 2010, the number of patients treated for ‘disc disease (M50, M51)’ has been increasing every year, and the number of patients per 100,000 population increased 1.7 times from 3,849 in 2006 to 4,496 in 2010. Medical expenses due to the ‘disc disease’ increased 1.56 times from KRW 438.7 billion in 2006 to KRW 686.9 billion in 2010, resulting in a huge medical cost for the ‘disc disease’.

Conservative therapies such as drug therapy and physical therapy are used to treat the low back pain caused by degenerative intervertebral discs, and when these conservative therapies are ineffective, surgical treatments such as intervertebral disc removal, spinal fusion, or artificial intervertebral disc insertion are considered. Although the surgical treatment has a short-term pain reduction effect, as a result of long-term follow-up, the lower back pain may worsen due to aggravation of the degenerative changes and induction of spinal instability. In other words, the existing surgical treatment is not a method to fundamentally treat the degenerative intervertebral discs, and there is a problem in that severe pain recurs in the long term.

Among biological therapies that have been attempted so far to solve the above problems, a method of supplementing insufficient growth factors is representative. Growth factors [transforming growth factor (TGF)β, insulin like growth factor-1, bone morphogenic protein-2] may be injected into a degenerated intervertebral disc to stimulate matrix production. However, since the injected growth factors are destroyed by in vivo degradation proteins over time, continuous injection is required, and the growth factors are produced through other animals, so the production cost is high.

In order to overcome the disadvantages, a research on a treatment in which the growth factors are constantly expressed through genetic manipulation has been conducted. The treatment is a therapeutic concept that obtains a biological effect by inserting a matrix assembly gene using a non-viral carrier and inducing continuous secretion of a desired protein in vivo, but it is difficult to apply directly to clinical practice.

Since the degenerative change in the intervertebral disc, the most common cause of chronic low back pain, is an irreversible disease for which there is no effective treatment method yet, it is required to develop a fundamental therapeutic agent.

### DISCLOSURE

### Technical Problem

In order to solve the problems, an aspect of the present invention provides a composition for regenerating nucleus pulposus and a composition for regenerating an intervertebral disc tissue for fundamental treatment of intervertebral disc diseases.

In addition, another aspect of the present invention provides a pharmaceutical composition for preventing or treating a vertebral diseases including the composition for regenerating nucleus pulposus or the composition for regenerating an intervertebral disc tissue.

### Technical Solution

A composition for regenerating nucleus pulposus according to an embodiment the present invention may include a fetal cartilage tissue-derived cell and a fetal cartilage tissue-derived extracellular matrix as active components.

A composition for regenerating an intervertebral disc tissue according to an embodiment the present invention may include a fetal cartilage tissue-derived cell and a fetal cartilage tissue-derived extracellular matrix as active components.

A pharmaceutical composition for preventing or treating a vertebral disease according to an embodiment of the present invention may include the composition for regenerating nucleus pulposus or the composition for regenerating an intervertebral disc tissue as an active component.

### Advantageous Effects

According to embodiments of the present invention, a composition for regenerating nucleus pulposus may simulate features of the nucleus pulposus, such as compression and tensile strength, to be regenerated into a tissue similar thereto, and a composition for regenerating an intervertebral disc tissue may be implanted into an intervertebral disc to restore an injured intervertebral disc tissue.

According to embodiments of the present invention, a pharmaceutical composition for preventing or treating a vertebral disease may be used for fundamental treatment for a vertebral disease such as a herniated intervertebral disc by including the composition for regenerating the nucleus pulposus or the composition for regenerating the intervertebral disc tissue having the features described above as an active component.

### BRIEF DESCRIPTIONS OF THE DRAWINGS

FIG. 1 illustrates schematically a method of treating an intervertebral disc using a composition according to an embodiment of the present invention.

FIG. 2 shows a process of making a degenerative lumbar intervertebral disc rabbit model by retroperitoneal approach according to an experimental example of the present invention.

FIG. 3 shows T MRI images of a lumbar intervertebral disc of a rabbit model of intervertebral disc injury according to an experimental example of the present invention.

FIG. 4 shows an embedding process for histological examination of a rabbit model of intervertebral disc injury according to an experimental example of the present invention.

FIG. 5 and FIG. 6 show histological examination results of the rabbit model of FIG. 4. FIG. 5 is a histological examination result of Rabbit No. 1, and FIG. 6 is a histological examination result of Rabbit No. 13.

### BEST MODE

Hereinafter, the present invention will be described in detail.

The present inventors have completed the present invention by confirming an nucleus pulposus regeneration effect of a composition including a fetal cartilage tissue-derived cell and a fetal cartilage tissue-derived extracellular matrix as active components in a rabbit lumbar intervertebral disc defect model while analyzing characteristics of the vertebral nucleus different from general articular cartilage and conduct a study on tissue regeneration accordingly.

An embodiment of the present invention provides a composition for regenerating nucleus pulposus including a fetal cartilage tissue-derived cell and a fetal cartilage tissue-derived extracellular matrix as active components.

The term “fetal cartilage tissue-derived cell” as used herein is a generic term for cells separated from fetal cartilage tissue, and preferably, chondrocytes isolated after the cartilage tissue is completely digested using collagenase or the like.

The term “fetal cartilage tissue-derived extracellular matrix” as used herein refers to a biopolymer aggregate composed of molecules synthesized by cells from the fetal cartilage tissue-derived cells, and secreted and accumulated extracellularly, and may include fibrous proteins such as collagen and elastin, complex proteins such as glycosaminoglycan (GAG), and cell adhesion proteins such as fibronectin and laminin. The extracellular matrix forms the shape of a tissue with the proteins described above and determines physical properties of the tissue, and plays an important role in maintaining an environment of the cell.

The term “spine” as used herein refers to a bone which maintains a main skeleton in humans from the neck down to the back, waist, hip, and tail, and consists of 33 vertebrae.

The term “nucleus pulposus” as used herein refers to a central part of an intervertebral disc which serves as a cushion between the vertebrae, and most of a healthy nucleus pulposus consists of water and is rich in proteoglycans capable of holding the water well.

The term “regeneration” as used herein refers to actions of supplementing, restoring, repairing, and recovering a defect of a tissue or an organ occurring due to various causes to make it the same tissue or organ as before, and may be used interchangeably with these terms.

A composition for regenerating nucleus pulposus according to an embodiment of the present invention may reproduce tissue characteristics of the nucleus pulposus. More specifically, the composition may simulate compressive and tensile strength of the nucleus pulposus, and may be in the form of a gel having fluidity and biomechanical stability, for example, the form of fetal cartilage tissue-derived extracellular matrix hydrogel loaded with the fetal cartilage tissue-derived cell, but is not limited thereto. The composition may have the biomechanical stability to withstand joint load, and may be deformed in a certain form according to vertebral joint motion.

The term “gel” as used herein refers to a jelly-like material and has physical properties ranging from soft and weak to strong and rough, and although the most of a gel is liquid, it acts like a solid as a whole due to its three-dimensional network structure. In Korean, “ [symbol] ” and “ [symbol] ” may be used interchangeably to indicate the gel.

An embodiment of the present invention provides a composition for regenerating an intervertebral disc tissue including a fetal cartilage tissue-derived cell and a fetal cartilage tissue-derived extracellular matrix as active components.

The term “intervertebral disc” as used herein refers to a cartilage structure which connects the vertebrae in the spine, and may be used interchangeably with “vertebral disc” or “spinal disc”. The intervertebral disc consists of the nucleus pulposus and an annulus fibrosus having blood vessels and nerves. The nucleus pulposus and the annulus fibrosus are structures distinct from each other, but they appear to be mixed at the boundary.

The composition for regenerating the intervertebral disc tissue according to the present disclosure may be in the form of fetal cartilage tissue-derived extracellular matrix hydrogel loaded with the fetal cartilage tissue-derived cell capable of reproducing the characteristics of the intervertebral disc tissue, but is not limited thereto.

The composition for regenerating the intervertebral disc tissue according to the present disclosure may restore an injured intervertebral disc tissue by being implanted in the intervertebral disc. According to an experimental example of the present invention, in the case that the composition was injected in a rabbit model of lumbar intervertebral disc injury, regeneration of the intervertebral disc tissue was confirmed.

An embodiment of the present invention provides a pharmaceutical composition for preventing or treating a verterbral disease including the composition for regenerating the nucleus pulposus or the composition for regenerating the intervertebral disc tissue as an active component.

For the pharmaceutical composition according to the present disclosure, the vertebral disease may include diseases caused by damage, injury, or defect in the vertebral cartilage tissue and the like due to a mechanical stimulation or inflammatory response, or diseases caused by degenerative changes due to aging, or genetic or environmental factors, and more specifically, may be one or more selected from the group consisting of lumbar herniated intervertebral disc, cervical herniated intervertebral disc, spinal sprain, spondylitis, and spinal stenosis, but is not limited thereto.

The composition may be transplanted into vertebral tissue and maintained for a long period of time, unlike conventional products which lose their function due to decomposition and require repeated administration.

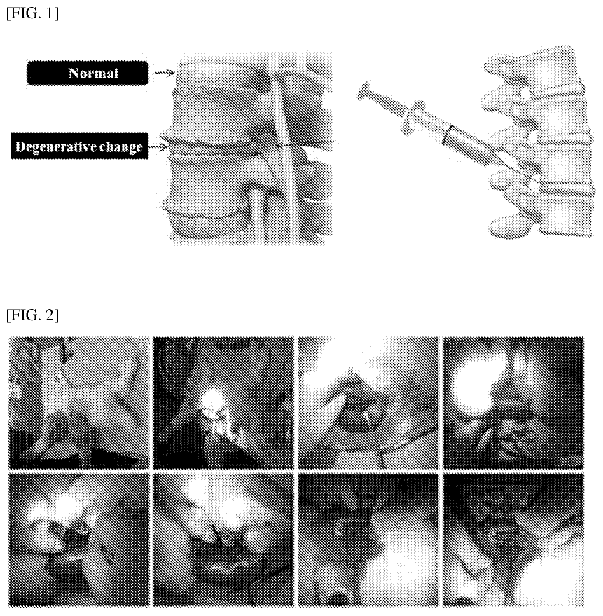

The pharmaceutical composition according to the present disclosure may be formulated in a form that can be directly injected into an injured or defected site of the vertebral tissue, for example, in the form of fetal cartilage tissue-derived extracellular matrix hydrogel loaded with differentiated fetal cartilage tissue-derived cells. FIG. 1 illustrates schematically a method of treating an intervertebral disc using the composition, and preferably, the composition may be prepared and implanted in the form of an injection or in a micro-invasive form, as shown in FIG. 1.

In addition, the injection may be prepared using aqueous solutions such as physiological saline, Ringel's solution, Hank's solution, or sterilized aqueous solution, and non-aqueous solutions such as vegetable oils, for example, olive oil, higher fatty acid esters such as ethyl oleic acid and ethanol, benzyl alcohol, propylene glycol, polyethylene glycol, or glycerin, and may use nonpenetrant solutions known in the art suitable for a barrier to pass through for mucosal penetration. It may further include a pharmaceutically acceptable carrier such as a stabilizer for prevention of deterioration such as ascorbic acid, sodium bisulfite, BHA, tocopherol, and EDTA, emulsifier, buffer for pH control, and preservative for preventing growth of microorganisms such as phenylmercuric nitrate, thimerosal, benzalkonium chloride, phenol, cresol, and benzyl alcohol.

The pharmaceutical composition according to the present disclosure may be administered in a pharmaceutically effective amount. The “pharmaceutically effective amount” refers to an amount sufficient to treat vertebral tissue injury at a reasonable benefit/risk ratio applicable to medical treatment and not to cause side effects.

An effective dose level of the pharmaceutical composition may be determined differently according to factors including a purpose of use, age, sex, weight and health status of a patient, the type of disease, severity, activity of the drug, sensitivity to the drug, an administration method, administration time, an administration route and excretion rate, treatment duration, and drugs used in combination or at the same time, and other factors well known in the medical field. The pharmaceutical composition according to the present disclosure may be implanted at an appropriate dose depending on the size of the injured or defected site of the vertebral tissue.

### MODES FOR CARRYING OUT INVENTION

Hereinafter, examples will be described in detail to help understanding of the present invention. The following examples are merely illustrative of the present invention, and the scope of the present invention is not limited to the following examples. The examples of the present invention are provided to more completely explain the present invention to those skilled in the art.

### EXAMPLE 1

### Separation and Culture of Chondrocytes Derived from Human Fetal Cartilage Tissue

For preparation of ArtiPaste, a composition for regenerating nucleus pulposus containing a fetal cartilage tissue-derived cell and a fetal cartilage tissue-derived extracellular matrix, chondrocytes were separated from a knee joint of a 12 to 15-week-old fetus. After washing cartilage tissue separated from the knee joint with phosphate buffered saline (PBS), the cartilage tissue was cultured for 4 hours in an incubator of 37° C., 5% CO₂ with Dulbecco's Modified Eagle Medium (hereinafter, DMEM) (Gibco, Grand Island, N.Y.) containing 0.2% (w/v) collagenase (Worthington Biochemical Corp., Lakewood, N.J.). Cartilage cells released after the cartilage tissue was completely digested were centrifuged at 1700 rpm for 10 minutes, and precipitated chondrocytes were seeded in a tissue culture dish (150 mm (dia.)×20 mm (h) at a density of 1×10⁶ cells per culture dish).

### EXAMPLE 2

### Preparation of ArtiPaste

The chondrocytes obtained in Example 1 were diluted with 10% fetal bovine serum (FBS), 50 units/mL penicillin, and 50 μg/mL streptomycin at 2×10⁵ cells in DMEM, and monolayer culture was performed for 15 to 18 days. After the culture, the medium was removed and 0.05% trypsin-EDTA (Gibco) was added to obtain cell membranes bound to extracellular matrix. To obtain cells and the cell membranes bound to the extracellular matrix, entire cell membranes including the cells and the extracellular matrix were obtained at once after treatment with 0.05% trypsin-EDTA (Gibco) without separating the cells with a pipet.

The obtained cell membranes including the cells and extracellular matrix were put in a 50 ml tube containing cartilage differentiation medium (Dulbecco's Modified Egle Medium-High Glucose (DMEM-HG) containing 1% antibiotic-antimycotic, 1.0 mg/mL insulin, 0.55 mg/mL human transferrin, 0.5 mg/mL sodium selenite, 50 μg/mL ascorbic acid, 1.25 mg/mL bovine serum albumin (BSA), 100 nM dexamethasone, 40 μg/mL proline, and 10 ng/ml TGF-β), and centrifuged at 250×g for 20 minutes to prepare a pellet-type structure.

ArtiPaste was prepared by putting the prepared cell pellet in a culture dish containing cartilage differentiation medium of the same composition as above, and culturing for 1 week, 2 weeks, and 3 weeks in an incubator at 37° C. and 5% CO₂.

### EXPERIMENTAL EXAMPLE 1

### Confirmation of Changes Due to Injection of ArtiPaste in Rabbit Model of Intervertebral Disc Injury

1. Method of Making Lumbar Intervertebral Disc Annulus Fibrosus Defect Model

FIG. 2 shows a process of making a degenerative lumbar intervertebral disc rabbit model by retroperitoneal approach according to an experimental example of the present invention. A non-clinical experiment on intervertebral disc regeneration treatment using the ArtiPaste for 15 New Zealand White Rabbits (females) weighing 2.5 kg or more (2.5 to 3 kg) was conducted with permission from the CHA University Institutional Animal Care and Use Committee.

Referring to FIG. 2, 15 mg/kg of Zoletil and 5 mg/kg of Rompun were mixed and injected into the rabbit's muscle, hair on the right side of the rabbit's lumbar region was removed, and after selecting a point about 3 cm to the right from the center of the lumbar region, the skin between the 13th rib and long bone was incised with a length of about 5 cm for surgery.

Taking care not to bleed, the fascia was incised and the lumbar intervertebral disc was directly exposed using the retroperitoneal approach for accessing lumbar vertebrae through muscles around the lumbar vertebra and peritoneum. A 16-gauge spinal needle was inserted into the intervertebral discs between lumbar vertebrae 3 and 4, 4 and 5, and 5 and 6 used in making of the model to a depth of 5 mm, resulting in defects in the annulus fibrosus of the intervertebral discs.

2-1. Insertion of ArtiPaste

Immediately after induction of the defects in the annulus fibrosus of the lumbar intervertebral disc, 50 μl of the ArtiPaste was directly injected into the intervertebral discs between the lumbar vertebrae 3 and 4, and 5 and 6. After injecting the ArtiPaste by injecting a 26-gauge spinal needle to a depth of 5 mm, leakage of the injected ArtiPaste was prevented using fibrin glue.

After all the animals were sacrificed 8 weeks after injection, MRI was taken and histological examination was performed. As an imaging index to evaluate a degree of degenerative changes in the intervertebral disc, T2-weighted images (repetition time/echo time; 2000/120 ms) through MRI were used.

2-2. Experimental Results

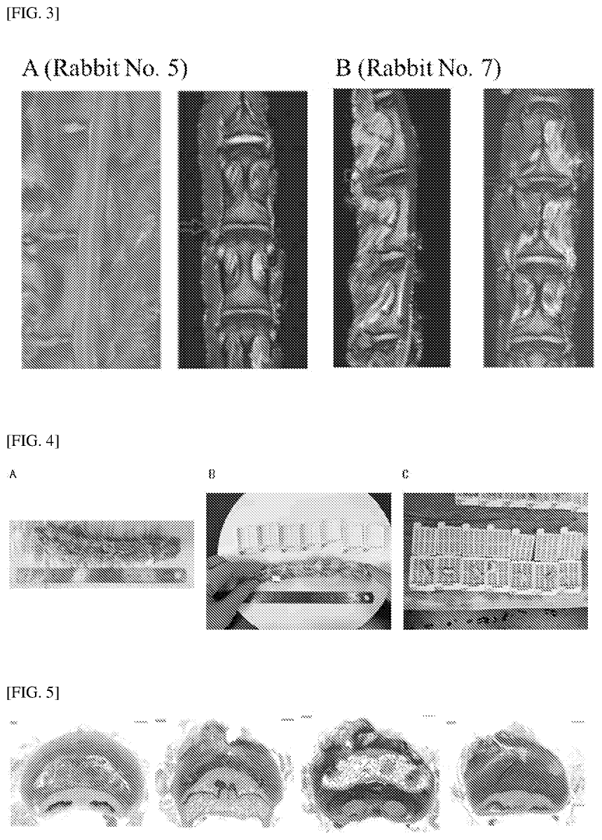

FIG. 3 shows T MRI images of a lumbar intervertebral disc of a rabbit model of intervertebral disc injury according to an experimental example of the present invention.

Referring to FIG. 3, after inducing annulus fibrosus defects in the intervertebral discs between the lumbar vertebrae 3 and 4, 4 and 5, and 5 and 6, the ArtiPaste was inserted only into the intervertebral discs between the lumbar vertebrae 3 and 4 and 5 and 6, and as a result, it was found that the intervertebral disc between the lumbar vertebrae 4 and 5, indicated by the red arrow, without any treatment, had a narrower gap and maintained the degenerative changes compared to the intervertebral discs between the lumbar vertebrae 3 and 4 and 5 and 6 with the ArtiPaste inserted. On the other hand, it was determined that the intervertebral discs between the lumbar vertebrae 3 and 4, and 5 and 6 with the ArtiPaste inserted were regenerated, compared to the intervertebral disc between the lumbar vertebrae 4 and 5 without any treatment.

3-1. Histological Examination

FIG. 4 shows an embedding process for histological examination of a rabbit model of intervertebral disc injury according to an experimental example of the present invention.

Referring to FIG. 4, A shows an appearance of a specimen, B shows the state of preparing the specimen to be cut centered on the intervertebral disc, and C shows the state in which the specimen has been cut centered on the intervertebral disc.

3-2. Test Results

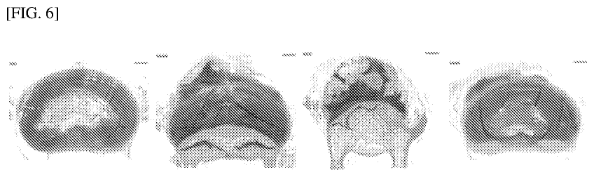

FIGS. 5 and 6 show histological examination results of the rabbit model of FIG. 4. FIG. 5 is a histological examination result of Rabbit No. 1, and FIG. 6 is a histological examination result of Rabbit No. 13.

Referring to FIGS. 5 and 6, the lumbar vertebrae 2-3 of Rabbit No. 1 of FIG. 5 are a normal control group, the lumbar vertebrae 3-4 are an ArtiPaste injection group, the lumbar vertebrae 4-5 are an ArtiPaste-free group, and the lumbar vertebrae 5-6 are an ArtiPaste injection group. It was found that defects of the annulus fibrosus of the intervertebral discs between the lumbar vertebrae 3 and 4, and 5 and 6 were restored.

Also in Rabbit No. 13 of FIG. 6, the lumbar vertebrae 2-3 are a normal control group, the lumbar vertebrae 3-4 are an ArtiPaste injection group, the lumbar vertebrae 4-5 are an ArtiPaste-free group, and the lumbar vertebrae 5-6 are an ArtiPaste injection group. It was found that defects of the annulus fibrous of the intervertebral discs between the lumbar vertebrae 3 and 4, and 5 and 6 were restored.

4. Assessment of Tissue Toxicity and Immune Response

In the case that the ArtiPaste was injected, no tissue toxicity was observed, no increase in inflammatory cells was observed, and tissue regeneration was observed compared to the intervertebral disc which was not administered with the ArtiPaste.

### COMPARATIVE EXAMPLE 1

### Comparison of ArtiPaste and GelStix (Replication Medical Inc)

The existing product, Gel Stix, contains mesenchymal stem cells or biomaterials such as hydrogel in the cells, and may exhibit a short-term function. However, it loses its function due to decomposition and requires repeated administration.

In contrast, the ArtiPaste according to an example embodiment of the present invention includes cartilage-derived stem cells and matrix, and may be regenerated into the similar tissue by simulating the compression and tensile strength of the nucleus pulposus. In other words, the ArtiPaste is a gel-type composition with fluidity and biomechanical safety, and may reproduce the characteristics of nucleus pulposus cartilage tissue biologically and anatomically.

Although specific parts of the present invention have been described in detail above, it is clear for those skilled in the art that these specific descriptions are merely preferred embodiments and the scope of the invention is not limited thereby. In other words, the substantial scope of the present invention is defined by the appended claims and their equivalents.

1-6. (canceled)

7. A method of regenerating nucleus pulposus comprising administering an effective amount of a composition comprising a fetal cartilage tissue-derived cell and a fetal cartilage tissue-derived extracellular matrix as active components to a subject in need of regenerating the nucleus pulposus.

8. The method of claim 7, wherein the composition simulates compressive and tensile strength of the nucleus pulposus and is in a form of a gel having fluidity and biomechanical stability.

9. A method of regenerating an intervertebral disc tissue comprising administering an effective amount of a composition comprising a fetal cartilage tissue-derived cell and a fetal cartilage tissue-derived extracellular matrix as active components to a subject in need of regenerating the intervertebral disc tissue.

10. The method of claim 9, wherein the composition is implanted in an intervertebral disc to restore an injured intervertebral disc tissue.

11. A method of preventing or treating a vertebral disease comprising administering a pharmaceutically effective amount of a pharmaceutical composition comprising a fetal cartilage tissue-derived cell and a fetal cartilage tissue-derived extracellular matrix as active components to a subject in need of preventing or treating the vertebral disease.

12. The method of claim 11, wherein the vertebral disease is at least one selected from the group consisting of lumbar herniated intervertebral disc, cervical herniated intervertebral disc, spinal sprain, spondylitis, and spinal stenosis.
